(19) United States
(12) Patent Application Publication
(10) Pub. No.: US 2021/0346276 A1
(43) Pub. Date: Nov. 11, 2021

(54) SKINCARE COMPOSITIONS CONTAINING CANNABIDIOL

(71) Applicant: GM Pharmaceuticals, Inc., Arlington, TX (US)

(72) Inventor: Odes W. Mitchell, Arlington, TX (US)

(21) Appl. No.: 17/308,459

(22) Filed: May 5, 2021

Related U.S. Application Data
(60) Provisional application No. 63/020,439, filed on May 5, 2020.

(51) Int. Cl. A61K 8/9789; A61K 8/34; A61K 8/37; A61K 8/891; A61K 31/05; A61K 8/9794; A61K 8/67; A61K 8/92; A61K 8/64; A61Q 19/08; A61Q 19/00; A61K 8/98

(52) U.S. Cl. CPC A61K 8/9789; A61K 8/342; A61K 8/37; A61K 8/345; A61K 8/891; A61K 31/05; A61K 8/987; A61K 8/9794; A61K 8/678; A61K 8/922; A61K 8/645; A61Q 19/08; A61Q 19/007; A61K 8/347

(57) ABSTRACT

In an embodiment, the present disclosure relates to a skincare composition including full spectrum hemp extract and a constituent. In some embodiments, the constituent includes at least one of an emollient, a moisturizer, a humectant, a perfuming agent, a skin soothing agent, a muscle relaxant, an antioxidant, a skin conditioner, a skin hydrator, or a skin nourishing agent. In a further embodiment, the present disclosure relates to a method that can include applying a skincare composition to an area of skin. In some embodiments, the skincare composition includes full spectrum hemp extract and a constituent that can include, without limitation, an emollient, a moisturizer, a humectant, a perfuming agent, a skin soothing agent, a muscle relaxant, an antioxidant, a skin conditioner, a skin hydrator, or a skin nourishing agent.

### CROSS-REFERENCE TO RELATED APPLICATIONS

This patent application claims priority from, and incorporates by reference the entire disclosure of, U.S. Provisional Patent Application No. 63/020,439 filed on May 5, 2020.

### TECHNICAL FIELD

The present disclosure relates generally to skincare compositions and more particularly, but not by way of limitation, to compositions and methods for use of skincare compositions containing cannabidiol.

### BACKGROUND

This section provides background information to facilitate a better understanding of the various aspects of the disclosure. It should be understood that the statements in this section of this document are to be read in this light, and not as admissions of prior art.

Skincare is a range of various practices that support skin integrity, enhance the appearance of skin, and relieve various conditions either on the skin (e.g., dry skin) or via application through the skin (e.g., muscle aches). Typically, skincare is practiced using several different types of products that can be applied to the skin, and can include, for example, lotions, creams, rubs, balms, ointments, and combinations of the same and like. Recently, cannabidiol (CBD), and other constituents of Cannabis sativa indica and cannabis hybrids having less than 0.3% tetrahydrocannabinol (THC) on a dry weight basis, have been shown to exhibit many advantageous health benefits, and these benefits can be particularly useful in routine and non-routine skincare practices (e.g., skin moisturizing and/or muscle rubs). As such, in some aspects, the present disclosure generally relates to skincare compositions with CBD and other constituents of Cannabis sativa.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure relates to a skincare composition including full spectrum hemp extract and a constituent. In some embodiments, the constituent includes at least one of an emollient, a moisturizer, a humectant, a perfuming agent, a skin soothing agent, a muscle relaxant, an antioxidant, a skin conditioner, a skin hydrator, a pain reliever, an anti-inflammatory, or a skin nourishing agent.

In an additional embodiment, the present disclosure relates to a skincare composition including full spectrum hemp extract present in a range of about 0.1 to about 5% (w/w) of the skincare composition and a skin conditioning agent present in a range of about 0.1 to about 15% (w/w) of the skincare composition. In some embodiments, the skin conditioning agent can include, without limitation, oils, extracts, plant extracts, plant oils, leaf oils, leaf extracts, seed oils, seed extracts, waxes, butters, isopropyl myristate, dimethicone, jojoba oil, vegetable stearic acid, Chamomilla recutita (Matricaria) flower extract in glycerin, shea butter, sunflower wax, Chamomilla recutita (Matricaria) flower oil, Carapa guaianensis (andiroba) seed oil, Rosmarinus officinalis (rosemary) leaf oil, Mentha piperita (peppermint) oil, Capsicum frutescens resin in sweet almond oil, Copernicia cerifera (carnauba) wax, frankincense oil, phenoxyethanol, butylated hydroxytoluene, camphor, and combinations thereof. In some embodiments, the skincare composition further includes an emollient present in a range of about 0.75 to about 75% (w/w) of the skincare composition. In some embodiments, the emollient can include, without limitation, plant oils, mineral oil, cocoa butter, petrolatum, fatty acids, animal oils, emu oil, mink oil, lanolin, argan oil, avocado oil, octyldodecanol, isopropyl myristate, dimethicone, cetyl palmitate, jojoba oil, shea butter, sunflower wax, medium-chain triglyceride (MCT) oil, beeswax, hemp seed oil, Carapa guaianensis (andiroba) seed oil, Copernicia cerifera (carnauba) wax, and combinations thereof.

In a further embodiment, the present disclosure relates to a method that can include applying a skincare composition to an area of skin. In some embodiments, the skincare composition includes full spectrum hemp extract and a constituent that can include, without limitation, an emollient, a moisturizer, a humectant, a perfuming agent, a skin soothing agent, a muscle relaxant, an antioxidant, a skin conditioner, a skin hydrator, a skin nourishing agent, and combinations thereof. In some embodiments, the skincare composition can include, without limitation, a body lotion, a face cream, a calming cream, a warming balm, a muscle rub, a deep muscle rub, and combinations thereof.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Cannabidiol (CBD) is a phytocannabinoid and is one of more than 113 identified cannabinoids in cannabis plants. CBD can account for up to 99% of the plant's extract. CBD is considered a safe, non-addictive substance, and is closely related to a medicinally active phytocannabinoid, tetrahydrocannabinol (THC). Both CBD and THC have significant therapeutic attributes. However, unlike THC, CBD is considered non-intoxicating.

One of the ways that CBD and THC interact with the body is via the endocannabinoid system (ECS). The ECS is a biological system composed of endocannabinoids, which are endogenous lipid-based retrograde neurotransmitters that bind to cannabinoid receptors, and cannabinoid receptor proteins that are expressed throughout the vertebrate central nervous system (including the brain) and the peripheral nervous system. Endocannabinoids are defined as the endogenous ligands of cannabinoid receptors and a growing body of evidence has emerged relating to the role of the ECS in the regulation of several physiological conditions and numerous diseases. Humans naturally synthesize endocannabinoids, chemical compounds that activate the same receptors as THC.

The ECS is involved in regulating physiological and cognitive processes, including, but not limited to, fertility, pregnancy, pre- and post-natal development, various activity of the immune system, appetite, pain sensation, mood, and memory. Two primary cannabinoid receptors have been identified: cannabinoid receptor 1 (CB₁) and cannabinoid receptor 2 (CB₂). CB₁ receptors are found predominantly in the brain and nervous system, as well as in peripheral organs and tissues. CB₂ receptors are more abundant outside the nervous system and are found in peripheral organs (e.g., cells of the immune system). However, both CB₁ and CB₂ receptors can be found throughout the body. One mechanism of action is believed to be that CBD attaches to the CB₂ receptors, though various research has indicated that CBD may not attach to either the CB₁ or CB₂ receptors, but instead directs the body to use more of its own naturally produced cannabinoids.

Full spectrum hemp extract is a pure extract from Cannabis sativa, including CBD and other constituents of Cannabis sativa indica and cannabis hybrids, cannabigerol (CBG), cannabinol (CBN), cannabichromene (CBC), flavonoids, terpenes, and minimal amounts of THC (approximately 0.3% on a dry weight basis). Full spectrum hemp extract exhibits all of the characteristics of CBD with additional benefit resulting from interactions of CBG, CBN, CBC, flavonoids, and terpenes with the body and their synergistic effects with CBD. As skin is considered the largest organ in the human body, skin acts as a powerful molecular pathway. In this manner, topical treatments such as, creams, lotions, balms, muscle rubs, ointments, and the like, when applied directly to a location on the skin, are absorbed directly at the target site. This allows for active ingredients of the topical treatments, such as, for example, the various constituents of full spectrum hemp extract, to target the surrounding area where the topical treatment was applied. Research suggests the existence of the ECS in the skin, and furthermore, that the ECS is present in skin cells. The dermal layers in the skin are capable of producing endocannabinoids. Additionally, several skin functions are controlled, at least in part, by the local skin ECS.

Dysregulation of the ECS is thought to be associated with a variety of dermatological disorders, including, but not limited to, atopic dermatitis and psoriasis. CBD and other cannabinoids and terpenes found in full spectrum hemp extract have been shown to help reduce pain and inflammation when applied topically to elevate skin conditions or inflammation in muscles. Many common skin problems, such as acne, rashes, contact and atopic dermatitis (eczema), rosacea, seborrheic dermatitis, and psoriasis are caused by inflammatory skin conditions. As such, full spectrum hemp extracts are an ideal candidate to add to various body lotions, face creams, and calming creams. Additionally, the pain reduction and anti-inflammatory properties induced by full spectrum hemp extracts prove beneficial in warming balms and muscle rubs to help alleviate symptoms of, for example, inflammatory arthritis, rheumatoid arthritis, joint and bone inflammation, muscle bruising and inflammation, muscle injuries, including, but not limited to, strains, pulls, overuse, and the like.

Full spectrum hemp extract also has potent antioxidant effects, indicating it can neutralize free radicals that cause oxidative stress and contribute to the aging process, and also provide emollient and skin nourishing benefits, as full spectrum hemp extract can moisturize skin without clogging pores. These properties of full spectrum hemp extract, in conjunction with its anti-inflammatory properties, add to its benefit as a facial cream and body lotion. Additionally, due to the antioxidant effects, anti-inflammatory properties, emollient, and skin nourishing benefits, full spectrum hemp extract can be utilized in calming creams, warming balms, and muscles rubs to help alleviate redness, dryness, dullness and uneven texture of skin, muscle pain, inflammation, and the like. Based on the aforementioned properties of full spectrum hemp extract, and CBD in general, various skincare compositions, such as, for example, lotions, creams, balms, rubs, ointments, and the like can harness these advantageous properties as an emollient and skin nourishment component. As detailed above, constituents of full spectrum hemp extract exhibit properties that prove highly beneficial in soothing and hydrating skin, and as such, can help manage dry, itchy, or scaly skin, can manage or alleviate muscle pain, and provide antioxidant or anti-inflammatory benefits.

As such, in some embodiments, the present disclosure relates generally to compositions and methods of use thereof for skincare. In some embodiments, the skincare compositions can include, without limitation, lotions, body lotions, daily body lotions, facial creams, calming creams, warming balms, muscle rubs, and deep muscle rubs leveraging the various benefits of full spectrum hemp extract. Example benefits can include, among others, reducing pain and inflammation, providing antioxidant effects, providing emollient and skin nourishing benefits, softening or soothing of skin, and combinations of the same and like.

In some embodiments, the full spectrum hemp extract can be present in the skincare compositions in an amount of approximately 0.1 to 5% (w/w) of the skincare composition. In some embodiments, the full spectrum hemp extract can be present in the skincare compositions in an amount of approximately 0.1 to 0.75% (w/w) of the skincare composition. In some embodiments, the full spectrum hemp extract can be present in the skincare composition in an amount of approximately 50 to 1,000 mg. In some embodiments, the full spectrum hemp extract can be present in the skincare composition in an amount of approximately 250 to 750 mg.

In some embodiments, the full spectrum hemp extract is present in the skincare composition in an amount of approximately 600 mg. In some embodiments, the full spectrum hemp extract is present in the skincare composition in an amount of approximately 250 mg. In some embodiments, the full spectrum hemp extract is present in the skincare composition in an amount of approximately 500 mg. In some embodiments, the full spectrum hemp extract is present in the skincare composition in an amount of approximately 750 mg.

In some embodiments, the full spectrum hemp extract is present in the skincare composition in an amount of approximately 0.45% (w/w) of the skincare composition. In some embodiments, the full spectrum hemp extract is present in the skincare composition in an amount of approximately 0.28% (w/w) of the skincare composition. In some embodiments, the full spectrum hemp extract is present in the skincare composition in an amount of approximately 0.23% (w/w) of the skincare composition. In some embodiments, the full spectrum hemp extract is present in the skincare composition in an amount of approximately 0.67% (w/w) of the skincare composition.

In some embodiments, the full spectrum hemp extract can also be combined with various constituents, such as, but not limited to, emollients, emulsifiers, moisturizers, humectants, perfuming agents, skin soothing agents, muscle relaxants, antioxidants, pH stabilizers, preservatives, skin conditioners, skin hydrators, skin nourishing agents, solvents, stabilizing agents, thickeners, film formers, essential oils, and combinations of the same and like.

In some embodiments, the emollients can soften dry, rough, flakey skin, and combinations thereof. In some embodiments, the emollients can include, without limitation, plant oils, mineral oil, cocoa butter, petrolatum, fatty acids, animal oils, such as, but not limited to, emu oil, mink oil, and lanolin, argan oil, avocado oil, and combinations of the same and like. In some embodiments, the emollients can include, without limitation, octyldodecanol, isopropyl myristate, dimethicone, cetyl palmitate, jojoba oil, shea butter, sunflower wax, medium-chain triglyceride (MCT) oil, beeswax, hemp seed oil, Carapa guaianensis (andiroba) seed oil, Copernicia cerifera (carnauba) wax, and combinations thereof.

In some embodiments, the emulsifiers can assist in the mixing of various constituent parts of the skincare composition that are otherwise immiscible or difficult to combine. In some embodiments, the emulsifiers can include, without limitation, anionic emulsifiers, cationic emulsifiers, nonionic emulsifiers, polymeric emulsifiers, and combinations of the same and like. In some embodiments, the emulsifiers can include, without limitation, glyceryl stearate, glyceryl stearate self-emulsifying (SE), PEG-100 stearate (polyethylene glycol ester of stearic acid), cetearyl olivate and sorbitan olivate (e.g., OLIVEM® 1000), and combinations thereof.

In some embodiments, the moisturizers can be utilized to lubricate, soften, protect, and hydrate the skin. In some embodiments, the moisturizers can include, without limitation, agents that draw water into the top layer of the skin, agents that fill cracks and seal water in the skin, agents that provide a protective barrier on the skin to prevent water loss, and combinations of the same and like. In some embodiments, the moisturizers can include, without limitation, oils, extracts, vitamins, waxes, and combinations of the same and like. In some embodiments, the moisturizers can include, without limitation, octyldodecanol, glycerin, Boswellia serrata resin extract, Lavandula angustifolia (lavender) oil, Mentha piperita (peppermint) oil, alpha-tocopheryl acetate (vitamin E), hydrolyzed soy protein, Aloe barbadensis leaf juice (Aloe vera extract), beeswax, and combinations thereof.

In some embodiments, the humectants can be utilized to reduce moisture loss. In some embodiments, the humectants can include, without limitation, alpha-hydroxy acids, salicylic acid, glycerin, hyaluronic acid, urea, panthenol, sodium lactate, glycols such as, for example, triethylene glycol, tripropylene glycol, propylene glycol, hexylene glycol, butylene glycol and polypropylene glycol, sugar alcohols (e.g., sorbitol), collagen, and combinations of the same and like. In some embodiments, the humectants can include, without limitation, glycerin, Boswellia serrata resin extract, Lavandula angustifolia (lavender) oil, Mentha piperita (peppermint) oil, and combinations thereof.

In some embodiments, the perfuming agents can be a single constituent or multiple constituents of fragrant oils, aroma compounds, fixatives and solvents, or the like, to provide fragrance to the skincare compositions disclosed herein. In some embodiments, the perfuming agents can include, without limitation, essential oils, plant extracts, flower extracts, flower oils, leaf oils, leaf extracts, and combinations of the same and like. In some embodiments, the perfuming agents can include, without limitation, Chamomilla recutita (Matricaria) flower extract in glycerin, Chamomilla recutita (Matricaria) flower oil, Lavandula angustifolia (lavender) oil, Rosmarinus officinalis (rosemary) leaf oil, Mentha piperita (peppermint) oil, Capsicum frutescens resin in sweet almond oil, frankincense oil, and combinations thereof.

In some embodiments, the skin soothing agents can be utilized to calm irritated or sensitive skin, reduce redness of irritated skin, visibly reduce signs of sensitive or irritated skin, and combinations of the same and like. In some embodiments, the skin soothing agents can include, without limitation, oils, extracts, plant oils, plant extracts, flower oils, flower extracts, leaf oils, leaf extracts, and combinations of the same and like. For example, Glycyrrhiza Glabra (licorice) root extract is a multi-functional botanical, including glycosides glycyrrhizin and glycyrrizinic acid, triterpene saponins, and flavonoids. Glycyrrhiza Glabra (licorice) root extract acts as a soothing ingredient and anti-inflammatory for relief of various skin conditions, such as, for example, eczema. As such, in some embodiments, Glycyrrhiza Glabra (licorice) root extract can be combined with full spectrum hemp extract to increase skin soothing and inflammatory relief caused by various skin conditions. In some embodiments, the skin soothing agents can include, without limitation, Aloe Barbadensis leaf juice (Aloe vera extract), Glycyrrhiza Glabra (licorice), and combinations thereof. In some embodiments, the skin soothing agent can include camphor.

In some embodiments, the muscle relaxants can be utilized to alleviate muscle spasms, pain, soreness, and combinations of the same and like. In some embodiments, the muscle relaxants can include, without limitation, menthol, gamma-aminobutyric acid, lidocaine, methyl salicylate, trolamine salicylate, and combinations of the same and like. In some embodiments, the muscle relaxants can include, without limitation, gamma-aminobutyric acid.

In some embodiments, the antioxidants can be utilized to inhibit oxidation producing free radicals that cause chain reactions that can potentially damage cells. In some embodiments, the antioxidants can be synthetic antioxidants or natural antioxidants. In some embodiments, the antioxidants can include, without limitation, flavonoids, flavones, catechins, carotenoids, vitamins, polyphenols, phytoestrogens, butylated hydroxyanisole, ethoxquin, propul gallate, and combinations of the same and like. In some embodiments, the antioxidants can include, without limitation, butylated hydroxytoluene, alpha-tocopheryl acetate (vitamin E), and combinations thereof.

In some embodiments, pH stabilizers can be utilized to maintain a particular pH or pH range of the skincare compositions disclosed herein. For example, in some embodiments, the pH stabilizers can include, without limitation, pH buffers, pH buffering systems, or other pH controlling compounds and/or systems, such as, for example, sodium hydroxide, calcium hydroxide, potassium hydroxide, borate-type buffering agents, N-cyclohexyl-2-aminoethanesulfonic acid (CHES), monosodium phosphate, calcium carbonate, sodium bicarbonate, disodium phosphate, bicarbonate buffering systems, phosphate buffering systems, alkaline buffering systems, acetate buffering systems, citric acid, citrate buffering systems, sodium-based buffering systems, salt-based buffering systems, and combinations of the same and like. In some embodiments, the pH stabilizers can include, without limitation, sodium citrate.

In some embodiments, the preservatives can be utilized to prevent growth of microbes, such as, for example, bacteria, mold, yeast, and combinations of the same and like. In some embodiments, the preservatives can include, without limitation, sorbic acid, sodium sorbate, sorbates, parabens, isothiazolinones (e.g., methylchloroisothiazolinone and/or benzisothiazolinone), formaldehyde releasers (e.g., DMDM hydantoin), hydantoins, sodium benzoate, benzoic acid, and combinations of the same and like. In some embodiments, the preservatives can include, without limitation, phenoxyethanol, butylated hydroxytoluene, and combinations thereof.

In some embodiments, the skincare compositions of the present disclosure can include skin conditioners to hydrate and soften skin. In some embodiments, the skin conditioners can include, without limitation, oils, extracts, plant extracts, plant oils, leaf oils, leaf extracts, seed oils, seed extracts, waxes, butters, and combinations of the same and like. In some embodiments, the skin conditioners can include, without limitation, isopropyl myristate, dimethicone, jojoba oil, vegetable stearic acid (e.g., EMERSOL® 7036), Chamomilla recutita (Matricaria) flower extract in glycerin, shea butter, sunflower wax, Chamomilla recutita (Matricaria) flower oil, Carapa guaianensis (andiroba) seed oil, Rosmarinus officinalis (rosemary) leaf oil, Mentha piperita (peppermint) oil, Capsicum frutescens resin in sweet almond oil, Copernicia cerifera (carnauba) wax, frankincense oil, phenoxyethanol, butylated hydroxytoluene, and combinations thereof.

In some embodiments, the skin hydrators can be utilized to hydrate the skin. In some embodiments, the skin hydrators can include, without limitation, alpha-hydroxy acids, salicylic acid, glycerin, hyaluronic acid, urea, panthenol, sodium lactate, glycols such as, for example, triethylene glycol, tripropylene glycol, propylene glycol, hexylene glycol, butylene glycol and polypropylene glycol, sugar alcohols (e.g., sorbitol), collagen, and combinations of the same and like. In some embodiments, the skin hydrators can include, without limitation, isosorbide dicaprylate.

In some embodiments, the solvents can include, without limitation, water, vegetable oils, animal oils, mineral oils, silicones, alcohols, and combinations of the same and like. In some embodiments, the solvents can include, without limitation, purified water. In some embodiments, each of the stabilizing agents, the thickeners, and the film formers can include, without limitation, agar, sodium alginate, carrageenans, guar, konjac, tragacanth, locust bean gum, psyllium, tara gum, fenugreek gum, xanthan gum, waxes, gels, and combinations of the same and like. In some embodiments, each of the stabilizing agents, the thickeners, and the film formers can include, without limitation, acrylates/acrylamide copolymer and mineral oil and polysorbate 85 (e.g., NOVEMER™ EC-1), vegetable stearic acid (e.g., EMERSOL® 7036), Copernicia cerifera (carnauba) wax, and combinations thereof.

In some embodiments, the skin nourishing agents can be utilized to nourish or soften, dry skin, flakey skin, greasy skin, aging skin, and combinations of the same and like. In some embodiments, the skin nourishing agents can include, without limitation, plant oils, mineral oil, shea butter, cocoa butter, petrolatum, fatty acids, animal oils, such as, but not limited to, emu oil, mink oil, and lanolin, and combinations of the same and like. In some embodiments, the skin nourishing agents can include, without limitation, octyldodecanol, isopropyl myristate, dimethicone, cetyl palmitate, jojoba oil, shea butter, sunflower wax, medium-chain triglyceride (MCT) oil, beeswax, hemp seed oil, Carapa guaianensis (andiroba) seed oil, Copernicia cerifera (carnauba) wax, and combinations thereof.

In some embodiments, the emollients can be present in the skincare composition in an amount of approximately 0.75 to 75% (w/w) of the skincare composition. In some embodiments, the emulsifiers can be present in the skincare composition in an amount of approximately 0.50 to 5% (w/w) of the skincare composition. In some embodiments, the moisturizers can be present in the skincare composition in an amount of approximately 0.01 to 30% (w/w) of the skincare composition. In some embodiments, the humectants can be present in the skincare composition in an amount of approximately 0.05 to 10% (w/w) of the skincare composition. In some embodiments, the perfuming agents can be present in the skincare composition in an amount of approximately 0.10 to 10% (w/w) of the skincare composition.

In some embodiments, the skin soothing agents can be present in the skincare composition in an amount of approximately 0.01 to 5% (w/w) of the skincare composition. In some embodiments, the muscle relaxants can be present in the skincare composition in an amount of approximately 0.01 to 5% (w/w) of the skincare composition. In some embodiments, the antioxidants can be present in the skincare composition in an amount of approximately 0.01 to 5% (w/w) of the skincare composition. In some embodiments, the pH stabilizers can be present in the skincare composition in an amount of approximately 0.01 to 5% (w/w) of the skincare composition. In some embodiments, the preservatives can be present in the skincare composition in an amount of approximately 0.01 to 5% (w/w) of the skincare composition.

In some embodiments, the skin conditioners can be present in the skincare composition in an amount of approximately 0.1 to 15% (w/w) of the skincare composition. In some embodiments, the skin hydrators can be present in the skincare composition in an amount of approximately 1 to 5% (w/w) of the skincare composition. In some embodiments, the skin nourishing agents can be present in the skincare composition in an amount of approximately 1 to 75% (w/w) of the skincare composition. In some embodiments, the solvents can be present in the skincare composition in an amount of approximately 35 to 95% (w/w) of the skincare composition. In some embodiments, the stabilizing agents can be present in the skincare composition in an amount of approximately 0.50 to 5% (w/w) of the skincare composition. In some embodiments, the thickeners can be present in the skincare composition in an amount of approximately 0.50 to 5% (w/w) of the skincare composition. In some embodiments, the film formers can be present in the skincare composition in an amount of approximately 1 to 5% (w/w) of the skincare composition.

In some embodiments, various constituents can have a combination of multiple properties (e.g., multi-class constituents), including, but not limited to, emollients and skin nourishing agents, moisturizers and emollients, moisturizers and humectants, moisturizers and perfuming agents, moisturizers and skin soothing agents, antioxidants and moisturizers, preservatives and antioxidants, skin conditioning agents and emollients, skin conditioning and perfuming agents, stabilizing agents, thickeners and skin conditioning agents, thickener and film formers, and thickeners and stabilizers.

In some embodiments, the emollients and skin nourishing agents can be present in the skincare composition in an amount of approximately 1 to 75% (w/w) of the skincare composition. In some embodiments, the moisturizers and emollients can be present in the skincare composition in an amount of approximately 1 to 30% (w/w) of the skincare composition. In some embodiments, the moisturizers and humectants can be present in the skincare composition in an amount of approximately 0.05 to 10% (w/w) of the skincare composition. In some embodiments, the moisturizers and perfuming agents can be present in the skincare composition in an amount of approximately 0.01 to 5% (w/w) of the skincare composition. In some embodiments, the moisturizers and skin soothing agents can be present in the skincare composition in an amount of approximately 0.01 to 5% (w/w) of the skincare composition.

In some embodiments, the antioxidants and moisturizers can be present in the skincare composition in an amount of approximately 0.01 to 5% (w/w) of the skincare composition. In some embodiments, the preservatives and antioxidants can be present in the skincare composition in an amount of approximately 0.01 to 5% (w/w) of the skincare composition. In some embodiments, the skin conditioning agents and emollients can be present in the skincare composition in an amount of approximately 0.01 to 15% (w/w) of the skincare composition. In some embodiments, the skin conditioning and perfuming agents can be present in the skincare composition in an amount of approximately 0.01 to 10% (w/w) of the skincare composition. In some embodiments, the stabilizing agents, thickeners and skin conditioning agents can be present in the skincare composition in an amount of approximately 1 to 10% (w/w) of the skincare composition. In some embodiments, the thickeners and film formers can be present in the skincare composition in an amount of approximately 0.01 to 5% (w/w) of the skincare composition. In some embodiments, the thickeners and stabilizers can be present in the skincare composition in an amount of approximately 0.01 to 5% (w/w) of the skincare composition.

### Working Examples

Reference will now be made to more specific embodiments of the present disclosure and data that provides support for such embodiments. However, it should be noted that the disclosure below is for illustrative purposes only and is not intended to limit the scope of the claimed subject matter in any way.

In an embodiment, as represented in Table 1 below, the present disclosure relates to a body lotion having full spectrum hemp extract. In various embodiments, the body lotion can be a topical preparation adaptable to be applied to skin. In some embodiments, the body lotion disclosed herein can be utilized to smooth, moisturize, and soften skin. Additionally, the body lotion can be utilized to provide antioxidants to the skin.

TABLE 1

| Ingredient | Concentration (w/w) | Function(s) |
|---|---|---|
| Purified Water | 80.97% | Solvent |
| Octyldodecanol | 3.00% | Moisturizer and Emollients |
| Isopropyl Myristate | 3.00% | Skin Conditioning and |
| | | Emollients |
| Glycerin | 2.00% | Moisturizer and Humectant |
| Dimethicone | 2.00% | Skin Conditioning |
| | | and Emollients |
| Glyceryl Stearate SE | 2.00% | Emulsifier |
| PEG-100 Stearate | 1.00% | Emulsifier |
| Cetyl Palmitate | 1.50% | Emollients |
| Phenoxyethanol | 0.80% | Preservative |
| NOVEMER ™ EC-1 | 2.50% | Thickener and Stabilizer |
| Full Spectrum Hemp | 0.45% | Emollient and |
| Extract | | Nourishing Skin |
| Boswellia serrata | 0.40% | Moisturizer |
| Resin Extract | | and Humectant |
| Lavandula angustifolia | 0.20% | Moisturizer |
| (Lavender) Oil | | and Humectant |
| Mentha piperita | 0.10% | Moisturizer |
| (Peppermint) Oil | | and Humectant |
| Butylated | 0.08% | Preservative and |
| Hydroxytoluene | | Antioxidant |

In some embodiments, the full spectrum hemp extract is present in the body lotion in an amount of approximately 600 mg. In some embodiments, the full spectrum hemp extract can be combined with various constituents, such as, but not limited to, emollients, emulsifiers, moisturizers, humectants, perfuming agents, skin soothing agents, muscle relaxants, antioxidants, pH stabilizers, preservatives, skin conditioners, skin hydrators, skin nourishing agents, solvents, stabilizing agents, thickeners, film formers, essential oils, and combinations of the same and like.

In this particular embodiment, purified water is present in an amount of about 80.97% (w/w), octyldodecanol is present in an amount of about 3.00% (w/w), isopropyl myristate is present in an amount of about 3.00% (w/w), glycerin is present in an amount of about 2.00% (w/w), dimethicone is present in an amount of about 2.00% (w/w), glyceryl stearate SE is present in an amount of about 2.00% (w/w), PEG-100 stearate is present in an amount of about 1.00% (w/w), cetyl palmitate is present in an amount of about 1.50% (w/w), phenoxyethanol is present in an amount of about 0.80% (w/w), NOVEMER™ EC-1 (acrylates/acrylamide copolymer and mineral oil and polysorbate 85) is present in an amount of about 2.50% (w/w), full spectrum hemp extract is present in an amount of about 0.45% (w/w), Boswellia serrata resin extract is present in an amount of about 0.40% (w/w), Lavandula angustifolia (lavender) oil is present in an amount of about 0.20% (w/w), Mentha piperita (peppermint) oil is present in an amount of about 0.10% (w/w), and butylated hydroxytoluene is present in an amount of about 0.08% (w/w) of the body lotion.

In some embodiments, a combination of the PEG-100 stearate and the glyceryl stearate SE provide for an enhanced emulsifying system that emulsify a broad range of emollients and oils. In addition, in some embodiments, the combination of PEG-100 stearate and glyceryl stearate SE provides for a nice feel to the skin. In some embodiments, a combination of the Lavandula angustifolia (lavender) oil and the Mentha piperita (peppermint) oil provide for a unique sensory and cooling effect on the skin.

In another embodiment, as represented in Table 2 below, the present disclosure relates to a face cream having full spectrum hemp extract. In various embodiments, the face cream can be a topical preparation to be applied to the face. In some embodiments, the face cream can moisturize, smoothen, and soften facial skin. Moreover, in some embodiments, the face cream can be utilized to provide antioxidants to facial skin.

TABLE 2

| Ingredient | Concentration (w/w) | Function(s) |
|---|---|---|
| Purified Water | 76.20% | Solvent |
| Octyldodecanol | 6.00% | Moisturizer and Emollients |
| Glycerin | 3.00% | Moisturizer and Humectant |
| Isosorbide Dicaprylate | 2.00% | Skin Hydrator |
| OLIVEM ® 1000 | 2.25% | Emulsifier |
| Jojoba Oil | 1.50% | Skin Conditioning |
| | | and Emollients |
| Glyceryl Stearate SE | 1.50% | Emulsifier |
| EMERSOL ® 7036 | 2.25% | Stabilizing Agent, |
| | | Thickener, and |
| | | Skin Conditioning |
| Phenoxyethanol | 0.75% | Preservative |
| NOVEMER ™ EC-1 | 1.50% | Thickener and Stabilizer |
| Alpha-Tocopheryl | 0.50% | Natural Antioxidant and |
| Acetate (Vitamin E) | | Moisturizer |
| Chamomilla recutita | 1.50% | Skin Conditioning |
| (Matricaria) Flower | | and Perfuming |
| Extract 20% in Glycerin | | |
| Full Spectrum | 0.28% | Emollient and |
| Hemp Extract | | Nourishing Skin |
| Glycyrrhiza glabra | 0.30% | Skin Soothing |
| (Licorice) Root Extract | | |
| Butylated | 0.08% | Preservative and |
| Hydroxytoluene | | Antioxidant |
| Sodium Citrate | 0.06% | pH Stabilizer |
| Hydrolyzed Soy Protein | 0.10% | Moisturizer |
| Aloe barbadensis | 0.10% | Moisturizer and |
| Leaf Juice (Aloe vera | | Skin Soothing |
| Extract 100:1) | | |

In some embodiments, the full spectrum hemp extract is present in the face cream in an amount of approximately 250 mg. In some embodiments, the full spectrum hemp extract can be combined with various constituents, such as, but not limited to, emollients, emulsifiers, moisturizers, humectants, perfuming agents, skin soothing agents, muscle relaxants, antioxidants, pH stabilizers, preservatives, skin conditioners, skin hydrators, skin nourishing agents, solvents, stabilizing agents, thickeners, film formers, essential oils, and combinations of the same and like.

In this particular embodiment, purified water is present in an amount of about 76.20% (w/w), octyldodecanol is present in an amount of about 6.00% (w/w), glycerin is present in an amount of about 3.00% (w/w), isosorbide dicaprylate is present in an amount of about 2.00% (w/w), OLIVEM® 1000 (cetearyl olivate and sorbitan olivate) is present in an amount of about 2.25% (w/w), jojoba oil is present in an amount of about 1.50% (w/w), glyceryl stearate SE is present in an amount of about 1.50% (w/w), EMERSOL® 7036 (vegetable stearic acid) is present in an amount of about 2.25% (w/w), phenoxyethanol is present in an amount of about 0.75% (w/w), NOVEMER™ EC-1 (acrylates/acrylamide copolymer and mineral oil and polysorbate 85) is present in an amount of about 1.50% (w/w), alpha-tocopheryl acetate (vitamin E) is present in an amount of about 0.50% (w/w), Chamomilla recutita (Matricaria) flower extract 20% in glycerin is present in an amount of about 1.50% (w/w), full spectrum hemp extract is present in an amount of about 0.28% (w/w), Glycyrrhiza Glabra (licorice) root extract is present in an amount of about 0.30% (w/w), butylated hydroxytoluene is present in an amount of about 0.08% (w/w), sodium citrate is present in an amount of about 0.06% (w/w), hydrolyzed soy protein is present in an amount of about 0.10% (w/w), and Aloe barbadensis leaf juice (Aloe vera extract 100:1) is present in an amount of about 0.10% (w/w) of the face cream.

In a further embodiment, as represented in Table 3 below, the present disclosure relates to a calming cream having full spectrum hemp extract. In various embodiments, the calming cream can be utilized to reduce redness, inflammation, and irritation of the skin. Additionally, the calming cream can be utilized, in some embodiments, to further moisturize and condition the skin to prevent future irritation. In various embodiments, the calming cream can be a topical preparation adaptable to be applied to skin.

TABLE 3

| | Concentration | |
|---|---|---|
| Ingredient | (w/w) | Function(s) |
| Purified Water | 70.00% | Solvent |
| Octyldodecanol | 7.00% | Moisturizer and Emollients |
| Glycerin | 4.00% | Moisturizer and Humectant |
| Shea Butter | 2.20% | Skin Conditioning |
| | | and Emollients |
| Sunflower Wax | 2.50% | Skin Conditioning |
| | | and Emollients |
| Glyceryl Stearate SE | 2.60% | Emulsifier |
| EMERSOL ® 7036 | 3.00% | Stabilizing Agent, |
| | | Thickener, and |
| | | Skin Conditioning |
| PEG-100 Stearate | 2.00% | Emulsifier |
| Cetyl Palmitate | 1.50% | Emollients |
| Phenoxyethanol | 0.75% | Preservative |
| NOVEMER ™ EC-1 | 2.60% | Thickener and Stabilizer |
| Chamomilla recutita | 0.20% | Skin Conditioning |
| (Matricaria) Flower Oil | | and Perfuming |
| Full Spectrum | 0.23% | Emollient and |
| Hemp Extract | | Nourishing Skin |
| Gamma-Aminobutyric | 0.30% | Muscle Relaxant |
| Acid | | |
| Lavandula angustifolia | 1.00% | Moisturizer and |
| (Lavender) Oil | | Perfuming |
| Butylated Hydroxytoluene | 0.08% | Preservative and |
| | | Antioxidant |
| Sodium Citrate | 0.10% | pH Stabilizer |
| Aloe barbadensis | 0.01% | Moisturizer and |
| Leaf Juice (Aloe vera | | Skin Soothing |
| Extract 100:1) | | |

In some embodiments, the full spectrum hemp extract is present in the calming cream in an amount of approximately 250 mg. In some embodiments, the full spectrum hemp extract can be combined with various constituents, such as, but not limited to, emollients, emulsifiers, moisturizers, humectants, perfuming agents, skin soothing agents, muscle relaxants, antioxidants, pH stabilizers, preservatives, skin conditioners, skin hydrators, skin nourishing agents, solvents, stabilizing agents, thickeners, film formers, essential oils, and combinations of the same and like.

In this particular embodiment, purified water is present in an amount of about 70.00% (w/w), octyldodecanol is present in an amount of about 7.00% (w/w), glycerin is present in an amount of about 4.00% (w/w), shea butter is present in an amount of about 2.20% (w/w), sunflower wax is present in an amount of about 2.50% (w/w), glyceryl stearate SE is present in an amount of about 2.60% (w/w), EMERSOL® 7036 (vegetable stearic acid) is present in an amount of about 3.00% (w/w), PEG-100 stearate is present in an amount of about 2.00% (w/w), cetyl palmitate is present in an amount of about 1.50% (w/w), phenoxyethanol is present in an amount of about 0.75% (w/w), NOVEMER™ EC-1 (acrylates/acrylamide copolymer and mineral oil and polysorbate 85) is present in an amount of about 2.60% (w/w), Chamomilla recutita (Matricaria) flower oil is present in an amount of about 0.20% (w/w), full spectrum hemp extract is present in an amount of about 0.23% (w/w), gamma-aminobutyric acid is present in an amount of about 0.30% (w/w), Lavandula angustifolia (lavender) oil is present in an amount of about 1.00% (w/w), butylated hydroxytoluene is present in an amount of about 0.08% (w/w), sodium citrate is present in an amount of about 0.10% (w/w), and Aloe barbadensis leaf juice (Aloe vera extract 100:1) is present in an amount of about 0.01% (w/w) of the calming cream.

In an additional embodiment, as represented in Table 4 below, the present disclosure relates to a warming balm having full spectrum hemp extract. In some embodiments, the warming balm of the present disclosure can be utilized to nourish skin, relieve irritation, and provide anti-inflammatory benefits to muscles. In some embodiments, the warming balm further moisturizes and conditions the skin. In some embodiments, the warming balm can be applied to the skin near muscles to relieve tension and sooth muscles. In some embodiments, the warming balm further relieves muscles soreness. In various embodiments, the warming balm can be a topical preparation adaptable to be applied to skin.

TABLE 4

| | Concentration | |
|---|---|---|
| Ingredient | (w/w) | Function(s) |
| MCT Oil | 49.65% | Emollient and Nourishing |
| | | Skin |
| Beeswax | 20.00% | Moisturizer and Emollients |
| Hemp Seed Oil | 8.00% | Emollient and Nourishing |
| | | Skin |
| Sunflower Wax | 5.00% | Skin Conditioning and |
| | | Emollients |
| Shea Butter | 5.00% | Skin Conditioning and |
| | | Emollients |
| Copernicia cerifera | 2.20% | Thickener and Film Former |
| (Carnauba) Wax | | |
| Carapa guaianensis | 3.00% | Skin Conditioning and |
| (Andiroba) Seed Oil | | Emollients |
| Rosmarinus officinalis | 4.00% | Skin Conditioning and |
| (Rosemary) Leaf Oil | | Perfuming |
| Mentha piperita | 1.80% | Skin Conditioning and |
| (Peppermint) Oil | | Perfuming |
| Full Spectrum | 0.45% | Emollient and |
| Hemp Extract | | Nourishing Skin |
| Capsicum frutescens Resin | 0.90% | Skin Conditioning and |
| in Sweet Almond Oil | | Perfuming |

In some embodiments, the full spectrum hemp extract is present in the warming balm in an amount of approximately 500 mg. In some embodiments, the full spectrum hemp extract can be combined with various constituents, such as, but not limited to, emollients, emulsifiers, moisturizers, humectants, perfuming agents, skin soothing agents, muscle relaxants, antioxidants, pH stabilizers, preservatives, skin conditioners, skin hydrators, skin nourishing agents, solvents, stabilizing agents, thickeners, film formers, essential oils, and combinations of the same and like.

In this particular embodiment, medium-chain triglyceride (MCT) oil is present in an amount of about 49.65% (w/w), beeswax is present in an amount of about 20.00% (w/w), hemp seed oil is present in an amount of about 8.00% (w/w), sunflower wax is present in an amount of about 5.00% (w/w), shea butter is present in an amount of about 5.00% (w/w), Copernicia cerifera (carnauba) wax is present in an amount of about 2.20% (w/w), Carapa Guaianensis (andiroba) seed oil is present in an amount of about 3.00% (w/w), Rosmarinus officinalis (rosemary) leaf oil is present in an amount of about 4.00% (w/w), Mentha piperita (peppermint) oil is present in an amount of about 1.80% (w/w), full spectrum hemp extract is present in an amount of about 0.45% (w/w), Capsicum frutescens resin in sweet almond oil is present in an amount of about 0.90% (w/w) of the warming balm. In some embodiments, the Capsicum frutescens resin in sweet almond oil provides for a warm feeling to the skin.

In an embodiment, as represented in Table 5 below, the present disclosure relates to a muscle rub having full spectrum hemp extract. In some embodiments, the muscle rub of the present disclosure can be utilized to nourish skin, relieve muscle pain and soreness, and provide anti-inflammatory benefits to muscles. In some embodiments, the muscle rub further moisturizes and conditions the skin. In some embodiments, the muscle rub can be applied to the skin near muscles to relieve tension and sooth muscles. In various embodiments, the muscle rub can be a topical preparation adaptable to be applied to skin.

Table 5

| | Concentration | |
|---|---|---|
| Ingredient | (w/w) | Function(s) |
| MCT Oil | 48.83% | Emollient and Nourishing Skin |
| Hemp Seed Oil | 17.00% | Emollient and Nourishing Skin |
| Beeswax | 14.00% | Moisturizer and Emollients |
| Sunflower Wax | 9.50% | Skin Conditioning and |
| | | Emollients |
| Shea Butter | 4.50% | Skin Conditioning and |
| | | Emollients |
| Copernicia cerifera | 3.50% | Skin Conditioning and |
| (Carnauba) Wax | | Emollients |
| Rosmarinus officinalis | 1.00% | Skin Conditioning and |
| (Rosemary) Leaf Oil | | Perfuming |
| Frankincense Oil | 0.50% | Skin Conditioning and |
| | | Perfuming |
| Lavandula angustifolia | 0.50% | Moisturizer and Perfuming |
| (Lavender) Oil | | |
| Full Spectrum | 0.67% | Emollient and Nourishing Skin |
| Hemp Extract | | |

In some embodiments, the full spectrum hemp extract is present in the muscle rub in an amount of approximately 750 mg. In some embodiments, the full spectrum hemp extract can be combined with various constituents, such as, but not limited to, emollients, emulsifiers, moisturizers, humectants, perfuming agents, skin soothing agents, muscle relaxants, antioxidants, pH stabilizers, preservatives, skin conditioners, skin hydrators, skin nourishing agents, solvents, stabilizing agents, thickeners, film formers, essential oils, and combinations of the same and like.

In this particular embodiment, medium-chain triglyceride (MCT) oil is present in an amount of about 48.83% (w/w), hemp seed oil is present in an amount of about 17.00% (w/w), beeswax is present in an amount of about 14.00% (w/w), sunflower wax is present in an amount of about 9.50% (w/w), shea butter is present in an amount of about 4.50% (w/w), Copernicia cerifera (carnauba) wax is present in an amount of about 3.50% (w/w), Rosmarinus officinalis (rosemary) leaf oil is present in an amount of about 1.00% (w/w), frankincense oil is present in an amount of about 0.50% (w/w), Lavandula angustifolia (lavender) oil is present in an amount of about 0.50% (w/w), and full spectrum hemp extract is present in an amount of about 0.67% (w/w) of the muscle rub.

In view of the aforementioned, in an embodiment, the present disclosure relates to a skincare composition including full spectrum hemp extract and a constituent. In some embodiments, the constituent includes at least one of an emollient, a moisturizer, a humectant, a perfuming agent, a skin soothing agent, a muscle relaxant, an antioxidant, a skin conditioner, a skin hydrator, or a skin nourishing agent.

In some embodiments, the full spectrum hemp extract is present in a range of 0.1 to 5% (w/w) of the skincare composition. In some embodiments, the constituent is an emollient present in a range of 0.75 to 75% (w/w) of the skincare composition. In some embodiments, the emollient can include, without limitation, plant oils, mineral oil, cocoa butter, petrolatum, fatty acids, animal oils, emu oil, mink oil, lanolin, argan oil, avocado oil, octyldodecanol, isopropyl myristate, dimethicone, cetyl palmitate, jojoba oil, shea butter, sunflower wax, medium-chain triglyceride (MCT) oil, beeswax, hemp seed oil, Carapa guaianensis (andiroba) seed oil, Copernicia cerifera (carnauba) wax, and combinations thereof. In some embodiments, the constituent is a moisturizer present in a range of 0.01 to 30% (w/w) of the skincare composition. In some embodiments, the moisturizer can include, without limitation, oils, extracts, vitamins, waxes, octyldodecanol, glycerin, Boswellia serrata resin extract, Lavandula angustifolia (lavender) oil, Mentha piperita (peppermint) oil, alpha-tocopheryl acetate (vitamin E), hydrolyzed soy protein, Aloe barbadensis leaf juice (Aloe vera extract), beeswax, and combinations thereof.

In some embodiments, the constituent is a humectant present in a range of 0.05 to 10% (w/w) of the skincare composition. In some embodiments, the humectant can include, without limitation, alpha-hydroxy acids, salicylic acid, glycerin, hyaluronic acid, urea, panthenol, sodium lactate, glycols, triethylene glycol, tripropylene glycol, propylene glycol, hexylene glycol, butylene glycol, polypropylene glycol, sugar alcohols, sorbitol, collagen, glycerin, Boswellia serrata resin extract, Lavandula angustifolia (lavender) oil, Mentha piperita (peppermint) oil, and combinations thereof. In some embodiments, the constituent is a perfuming agent present in a range of 0.10 to 10% (w/w) of the skincare composition. In some embodiments, the perfuming agent can include, without limitation, essential oils, plant extracts, flower extracts, flower oils, leaf oils, leaf extracts, Chamomilla recutita (Matricaria) flower extract in glycerin, Chamomilla recutita (Matricaria) flower oil, Lavandula angustifolia (lavender) oil, Rosmarinus officinalis (rosemary) leaf oil, Mentha piperita (peppermint) oil, Capsicum frutescens resin in sweet almond oil, frankincense oil, and combinations thereof.

In some embodiments, the constituent is a skin soothing agent present in a range of 0.01 to 5% (w/w) of the skincare composition. In some embodiments, the skin soothing agent can include, without limitation, oils, extracts, plant oils, plant extracts, flower oils, flower extracts, leaf oils, leaf extracts, Aloe Barbadensis leaf juice (Aloe vera extract), Glycyrrhiza Glabra (licorice), camphor, and combinations thereof. In some embodiments, the constituent is a muscle relaxant present in a range of 0.01 to 5% (w/w) of the skincare composition. In some embodiments, the muscle relaxant can include, without limitation, menthol, gamma-aminobutyric acid, lidocaine, methyl salicylate, trolamine salicylate, and combinations thereof. In some embodiments, the constituent is an antioxidant present in a range of 0.01 to 5% (w/w) of the skincare composition. In some embodiments, the antioxidant can include, without limitation, flavonoids, flavones, catechins, carotenoids, vitamins, polyphenols, phytoestrogens, butylated hydroxyanisole, ethoxquin, propul gallate, butylated hydroxytoluene, alpha-tocopheryl acetate (vitamin E), and combinations thereof.

In some embodiments, the constituent is a skin conditioner present in a range of 0.1 to 15% (w/w) of the skincare composition. In some embodiments, the skin conditioner can include, without limitation, oils, extracts, plant extracts, plant oils, leaf oils, leaf extracts, seed oils, seed extracts, waxes, butters, isopropyl myristate, dimethicone, jojoba oil, vegetable stearic acid, Chamomilla recutita (Matricaria) flower extract in glycerin, shea butter, sunflower wax, Chamomilla recutita (Matricaria) flower oil, Carapa guaianensis (andiroba) seed oil, Rosmarinus officinalis (rosemary) leaf oil, Mentha piperita (peppermint) oil, Capsicum frutescens resin in sweet almond oil, Copernicia cerifera (carnauba) wax, frankincense oil, phenoxyethanol, butylated hydroxytoluene, and combinations thereof.

In some embodiments, the constituent is a skin hydrator present in a range of 1 to 5% (w/w) of the skincare composition. In some embodiments, the skin hydrator can include, without limitation, alpha-hydroxy acids, salicylic acid, glycerin, hyaluronic acid, urea, panthenol, sodium lactate, glycols, triethylene glycol, tripropylene glycol, propylene glycol, hexylene glycol, butylene glycol, polypropylene glycol, sugar alcohols, sorbitol, collagen, isosorbide dicaprylate, and combinations thereof. In some embodiments, the constituent is a skin nourishing agent present in a range of 1 to 75% (w/w) of the skincare composition. In some embodiments, the skin nourishing agent can include, without limitation, plant oils, mineral oil, shea butter, cocoa butter, petrolatum, fatty acids, animal oils, emu oil, mink oil, lanolin, octyldodecanol, isopropyl myristate, dimethicone, cetyl palmitate, jojoba oil, shea butter, sunflower wax, medium-chain triglyceride (MCT) oil, beeswax, hemp seed oil, Carapa guaianensis (andiroba) seed oil, Copernicia cerifera (carnauba) wax, and combinations thereof. In some embodiments, the skincare composition further includes at least one of an emulsifier, a pH stabilizer, a preservative, a solvent, a stabilizing agent, a thickener, or a film former.

In some embodiments, the constituent is a plurality of constituents including a moisturizer, an emollient, a skin conditioner, a humectant, and an antioxidant. In some embodiments, the plurality of constituents is octyldodecanol, isopropyl myristate, glycerin, dimethicone, cetyl palmitate, Boswellia serrata resin extract, Lavandula angustifolia (lavender) oil, Mentha piperita (peppermint) oil, and butylated hydroxytoluene.

In some embodiments, the constituent is a plurality of constituents including a moisturizer, an emollient, a humectant, a skin hydrator, a skin conditioner, an antioxidant, a perfuming agent, and a skin soothing agent. In some embodiments, the plurality of constituents is octyldodecanol, glycerin, isosorbide dicaprylate, jojoba oil, vegetable stearic acid, alpha-tocopheryl acetate (vitamin E), Chamomilla recutita (Matricaria) flower extract in glycerin, Glycyrrhiza Glabra (licorice), butylated hydroxytoluenem, hydrolyzed soy protein, and Aloe barbadensis leaf juice (Aloe vera extract).

In some embodiments, the constituent is a plurality of constituents including a moisturizer, an emollient, a humectant, a skin conditioner, a perfuming agent, a muscle relaxant, an antioxidant, and a skin soothing agent. In some embodiments, the plurality of constituents is octyldodecanol, glycerin, shea butter, sunflower wax, vegetable stearic acid, cetyl palmitate, Chamomilla recutita (Matricaria) flower oil, gamma-aminobutyric acid, Lavandula angustifolia (lavender) oil, butylated hydroxytoluene, and Aloe barbadensis leaf juice (Aloe vera extract).

In some embodiments, the constituent is a plurality of constituents including an emollient, a skin nourishing agent, a moisturizer, a skin conditioner, and a perfuming agent. In some embodiments, the plurality of constituents is medium-chain triglyceride (MCT) oil, beeswax, hemp seed oil, sunflower wax, shea butter, Carapa guaianensis (andiroba) seed oil, Rosmarinus officinalis (rosemary) leaf oil, Mentha piperita (peppermint) oil, and Capsicum frutescens resin in sweet almond oil.

In some embodiments, the constituent is a plurality of constituents including an emollient, a skin nourishing agent, a moisturizer, a skin conditioner, and a perfuming agent. In some embodiments, the plurality of constituents is medium-chain triglyceride (MCT) oil, hemp seed oil, beeswax, sunflower wax, shea butter, Copernicia cerifera (carnauba) wax, Rosmarinus officinalis (rosemary) leaf oil, frankincense oil, and Lavandula angustifolia (lavender) oil.

In an additional embodiment, the present disclosure relates to a skincare composition including full spectrum hemp extract present in a range of 0.1 to 5% (w/w) of the skincare composition and a skin conditioning agent present in a range of 0.1 to 15% (w/w) of the skincare composition. In some embodiments, the skin conditioning agent can include, without limitation, oils, extracts, plant extracts, plant oils, leaf oils, leaf extracts, seed oils, seed extracts, waxes, butters, isopropyl myristate, dimethicone, jojoba oil, vegetable stearic acid, Chamomilla recutita (Matricaria) flower extract in glycerin, shea butter, sunflower wax, Chamomilla recutita (Matricaria) flower oil, Carapa guaianensis (andiroba) seed oil, Rosmarinus officinalis (rosemary) leaf oil, Mentha piperita (peppermint) oil, Capsicum frutescens resin in sweet almond oil, Copernicia cerifera (carnauba) wax, frankincense oil, phenoxyethanol, butylated hydroxytoluene, and combinations thereof. In some embodiments, the skincare composition further includes an emollient present in a range of 0.75 to 75% (w/w) of the skincare composition. In some embodiments, the emollient can include, without limitation, plant oils, mineral oil, cocoa butter, petrolatum, fatty acids, animal oils, emu oil, mink oil, lanolin, argan oil, avocado oil, octyldodecanol, isopropyl myristate, dimethicone, cetyl palmitate, jojoba oil, shea butter, sunflower wax, medium-chain triglyceride (MCT) oil, beeswax, hemp seed oil, Carapa guaianensis (andiroba) seed oil, Copernicia cerifera (carnauba) wax, and combinations thereof.

In a further embodiment, the present disclosure relates to a method that can include applying a skincare composition to an area of skin. In some embodiments, the skincare composition includes full spectrum hemp extract and a constituent that can include, without limitation, an emollient, a moisturizer, a humectant, a perfuming agent, a skin soothing agent, a muscle relaxant, an antioxidant, a skin conditioner, a skin hydrator, or a skin nourishing agent. In some embodiments, the skincare composition can include, without limitation, a body lotion, a face cream, a calming cream, a warming balm, a muscle rub, and combinations thereof.

In addition, in some embodiments the compositions of the present disclosure can further include, for example, various components to provide synergist effects and can include, without limitation, pain relievers and pain management constituents, choline salicylate, methyl salicylate, magnesium salicylate, salicylate salts, acetaminophen, ibuprofen, and combinations thereof. In some embodiments, the compositions of the present disclosure can include pain relief agents, naproxen, Capzasin, diclofenac sodium (Voltaren), histamine dihydrochoride, lidocaine, caffeine, butalbital, and combinations thereof.

While various embodiments of the present disclosure pertain to body lotions, face creams, calming creams, warming balms, muscle rubs, and various other skincare compositions, the combination of the various constituents of each are envisioned for use outside of skincare compositions.

The components of the compositions described in detail above exhibit excellent properties individually, and in combination with other constituents, for use in pain relief and pain management for example. Full spectrum hemp extract, as described above, includes CBD, CBG, CBN, CBC, flavonoids, and terpenes, and exhibits all of the characteristics of CBD with additional benefit resulting from interactions of CBG, CBN, CBC, flavonoids, and terpenes with the body and their synergistic effects with CBD. These benefits can be realized not only topically but also through the use of various pills, capsules, liquids, and the like to form oral compositions.

In general, oral dosing can be easier to deliver appropriate concentrations of each constituent and are often times highly convenient for the user. As such, various embodiments of the present disclosure pertain to full spectrum hemp extract (or any cannabidiol) in forms including, but not limited to, pills, capsules, liquids, and combinations thereof. In addition, various components can be added to provide synergist effects and can include, without limitation, pain relievers and pain management constituents. For example, full spectrum hemp extract can be combined with, for example, choline salicylate, methyl salicylate, magnesium salicylate, salicylate salts, acetaminophen, ibuprofen, and combinations thereof. In some embodiments, the full spectrum hemp extract can be combined with pain relief agents, naproxen, Capzasin, diclofenac sodium (Voltaren), histamine dihydrochoride, lidocaine, caffeine, butalbital, and combinations thereof.

In some embodiments, the oral compositions can include full spectrum hemp extract in the form of an oil in a capsule of soft gel capsule. In some embodiment, the oral compositions can include full spectrum hemp extract in powder form or crystalline powder form for use in a tablet or other solid pill form. In some embodiments, the oral compositions can include full spectrum hemp extract in a liquid form for liquid oral compositions.

Although various embodiments of the present disclosure have been described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A skincare composition comprising:
full spectrum hemp extract; and
a constituent,
wherein the constituent comprises at least one of an emollient, a moisturizer, a humectant, a perfuming agent, a skin soothing agent, a muscle relaxant, an antioxidant, a skin conditioner, a skin hydrator, or a skin nourishing agent.

2. The skincare composition of claim 1, wherein the full spectrum hemp extract is present in a range of 0.1 to 5% (w/w) of the skincare composition.

3. The skincare composition of claim 1, wherein the constituent comprises an emollient present in a range of 0.75 to 75% (w/w) of the skincare composition, and wherein the emollient is selected from the group consisting of plant oils, mineral oil, cocoa butter, petrolatum, fatty acids, animal oils, emu oil, mink oil, lanolin, argan oil, avocado oil, octyldodecanol, isopropyl myristate, dimethicone, cetyl palmitate, jojoba oil, shea butter, sunflower wax, medium-chain triglyceride (MCT) oil, beeswax, hemp seed oil, Carapa guaianensis (andiroba) seed oil, Copernicia cerifera (carnauba) wax, and combinations thereof.

4. The skincare composition of claim 1, wherein the constituent comprises a moisturizer present in a range of 0.01 to 30% (w/w) of the skincare composition, and wherein the moisturizer is selected from the group consisting of oils, extracts, vitamins, waxes, octyldodecanol, glycerin, Boswellia serrata resin extract, Lavandula angustifolia (lavender) oil, Mentha piperita (peppermint) oil, alpha-tocopheryl acetate (vitamin E), hydrolyzed soy protein, Aloe barbadensis leaf juice (Aloe vera extract), beeswax, and combinations thereof.

5. The skincare composition of claim 1, wherein the constituent comprises a humectant present in a range of 0.05 to 10% (w/w) of the skincare composition, and wherein the humectant is selected from the group consisting of alpha-hydroxy acids, salicylic acid, glycerin, hyaluronic acid, urea, panthenol, sodium lactate, glycols, triethylene glycol, tripropylene glycol, propylene glycol, hexylene glycol, butylene glycol, polypropylene glycol, sugar alcohols, sorbitol, collagen, glycerin, Boswellia serrata resin extract, Lavandula angustifolia (lavender) oil, Mentha piperita (peppermint) oil, and combinations thereof.

6. The skincare composition of claim 1, wherein the constituent comprises a perfuming agent present in a range of 0.10 to 10% (w/w) of the skincare composition, and wherein the perfuming agent is selected from the group consisting of essential oils, plant extracts, flower extracts, flower oils, leaf oils, leaf extracts, Chamomilla recutita (Matricaria) flower extract in glycerin, Chamomilla recutita (Matricaria) flower oil, Lavandula angustifolia (lavender) oil, Rosmarinus officinalis (rosemary) leaf oil, Mentha piperita (peppermint) oil, Capsicum frutescens resin in sweet almond oil, frankincense oil, and combinations thereof.

7. The skincare composition of claim 1, wherein the constituent comprises a skin soothing agent present in a range of 0.01 to 5% (w/w) of the skincare composition, and wherein the skin soothing agent is selected from the group consisting of oils, extracts, plant oils, plant extracts, flower oils, flower extracts, leaf oils, leaf extracts, Aloe Barbadensis leaf juice (Aloe vera extract), Glycyrrhiza Glabra (licorice), camphor, and combinations thereof.

8. The skincare composition of claim 1, wherein the constituent comprises a muscle relaxant present in a range of 0.01 to 5% (w/w) of the skincare composition, and wherein the muscle relaxant is selected from the group consisting of menthol, gamma-aminobutyric acid, lidocaine, methyl salicylate, trolamine salicylate, and combinations thereof.

9. The skincare composition of claim 1, wherein the constituent comprises an antioxidant present in a range of 0.01 to 5% (w/w) of the skincare composition, and wherein the antioxidant is selected from the group consisting of flavonoids, flavones, catechins, carotenoids, vitamins, polyphenols, phytoestrogens, butylated hydroxyanisole, ethoxquin, propul gallate, butylated hydroxytoluene, alpha-tocopheryl acetate (vitamin E), and combinations thereof.

10. The skincare composition of claim 1, wherein the constituent comprises a skin conditioner present in a range of 0.1 to 15% (w/w) of the skincare composition, and wherein the skin conditioner is selected from the group consisting of oils, extracts, plant extracts, plant oils, leaf oils, leaf extracts, seed oils, seed extracts, waxes, butters, isopropyl myristate, dimethicone, jojoba oil, vegetable stearic acid, Chamomilla recutita (Matricaria) flower extract in glycerin, shea butter, sunflower wax, Chamomilla recutita (Matricaria) flower oil, Carapa guaianensis (andiroba) seed oil, Rosmarinus officinalis (rosemary) leaf oil, Mentha piperita (peppermint) oil, Capsicum frutescens resin in sweet almond oil, Copernicia cerifera (carnauba) wax, frankincense oil, phenoxyethanol, butylated hydroxytoluene, and combinations thereof.

11. The skincare composition of claim 1, wherein the constituent comprises a skin hydrator present in a range of 1 to 5% (w/w) of the skincare composition, and wherein the skin hydrator is selected from the group consisting of alpha-hydroxy acids, salicylic acid, glycerin, hyaluronic acid, urea, panthenol, sodium lactate, glycols, triethylene glycol, tripropylene glycol, propylene glycol, hexylene glycol, butylene glycol, polypropylene glycol, sugar alcohols, sorbitol, collagen, isosorbide dicaprylate, and combinations thereof.

12. The skincare composition of claim 1, wherein the constituent comprises a skin nourishing agent present in a range of 1 to 75% (w/w) of the skincare composition, and wherein the skin nourishing agent is selected from the group consisting of plant oils, mineral oil, shea butter, cocoa butter, petrolatum, fatty acids, animal oils, emu oil, mink oil, lanolin, octyldodecanol, isopropyl myristate, dimethicone, cetyl palmitate, jojoba oil, shea butter, sunflower wax, medium-chain triglyceride (MCT) oil, beeswax, hemp seed oil, Carapa guaianensis (andiroba) seed oil, Copernicia cerifera (carnauba) wax, and combinations thereof.

13. The skincare composition of claim 1, further comprising at least one of an emulsifier, a pH stabilizer, a preservative, a solvent, a stabilizing agent, a thickener, or a film former.

14. The skincare composition of claim 1, wherein the constituent is a plurality of constituents comprising:
a moisturizer, an emollient, a skin conditioner, a humectant, and an antioxidant each independently selected from the group consisting of octyldodecanol, isopropyl myristate, glycerin, dimethicone, cetyl palmitate, Boswellia serrata resin extract, Lavandula angustifolia (lavender) oil, Mentha piperita (peppermint) oil, and butylated hydroxytoluene.

15. The skincare composition of claim 1, wherein the constituent is a plurality of constituents comprising:
a moisturizer, an emollient, a humectant, a skin hydrator, a skin conditioner, an antioxidant, a perfuming agent, and a skin soothing agent each independently selected from the group consisting of octyldodecanol, glycerin, isosorbide dicaprylate, jojoba oil, vegetable stearic acid, alpha-tocopheryl acetate (vitamin E), Chamomilla recutita (Matricaria) flower extract in glycerin, Glycyrrhiza Glabra (licorice), butylated hydroxytoluenem, hydrolyzed soy protein, and Aloe barbadensis leaf juice (Aloe vera extract).

16. The skincare composition of claim 1, wherein the constituent is a plurality of constituents comprising:
a moisturizer, an emollient, a humectant, a skin conditioner, a perfuming agent, a muscle relaxant, an antioxidant, and a skin soothing agent each independently selected from the group consisting of octyldodecanol, glycerin, shea butter, sunflower wax, vegetable stearic acid, cetyl palmitate, Chamomilla recutita (Matricaria) flower oil, gamma-aminobutyric acid, Lavandula angustifolia (lavender) oil, butylated hydroxytoluene, and Aloe barbadensis leaf juice (Aloe vera extract).

17. The skincare composition of claim 1, wherein the constituent is a plurality of constituents comprising:
an emollient, a skin nourishing agent, a moisturizer, a skin conditioner, and a perfuming agent each independently selected from the group consisting of medium-chain triglyceride (MCT) oil, beeswax, hemp seed oil, sunflower wax, shea butter, Carapa guaianensis (andiroba) seed oil, Rosmarinus officinalis (rosemary) leaf oil, Mentha piperita (peppermint) oil, and Capsicum frutescens resin in sweet almond oil.

18. The skincare composition of claim 1, wherein the constituent is a plurality of constituents comprising:
an emollient, a skin nourishing agent, a moisturizer, a skin conditioner, and a perfuming agent each independently selected from the group consisting of medium-chain triglyceride (MCT) oil, hemp seed oil, beeswax, sunflower wax, shea butter, Copernicia cerifera (carnauba) wax, Rosmarinus officinalis (rosemary) leaf oil, frankincense oil, and Lavandula angustifolia (lavender) oil.

19. A skincare composition comprising:
full spectrum hemp extract present in a range of 0.1 to 5% (w/w) of the skincare composition;
a skin conditioning agent present in a range of 0.1 to 15% (w/w) of the skincare composition,
wherein the skin conditioning agent is selected from the group consisting of oils, extracts, plant extracts, plant oils, leaf oils, leaf extracts, seed oils, seed extracts, waxes, butters, isopropyl myristate, dimethicone, jojoba oil, vegetable stearic acid, Chamomilla recutita (Matricaria) flower extract in glycerin, shea butter, sunflower wax, Chamomilla recutita (Matricaria) flower oil, Carapa guaianensis (andiroba) seed oil, Rosmarinus officinalis (rosemary) leaf oil, Mentha piperita (peppermint) oil, Capsicum frutescens resin in sweet almond oil, Copernicia cerifera (carnauba) wax, frankincense oil, phenoxyethanol, butylated hydroxytoluene, camphor, and combinations thereof; and
an emollient present in a range of 0.75 to 75% (w/w) of the skincare composition,
wherein the emollient is selected from the group consisting of plant oils, mineral oil, cocoa butter, petrolatum, fatty acids, animal oils, emu oil, mink oil, lanolin, argan oil, avocado oil, octyldodecanol, isopropyl myristate, dimethicone, cetyl palmitate, jojoba oil, shea butter, sunflower wax, medium-chain triglyceride (MCT) oil, beeswax, hemp seed oil, Carapa guaianensis (andiroba) seed oil, Copernicia cerifera (carnauba) wax, and combinations thereof.

20. A method comprising:
applying a skincare composition to an area of skin, wherein the skincare composition comprises:
full spectrum hemp extract; and
a constituent selected from the group consisting of an emollient, a moisturizer, a humectant, a perfuming agent, a skin soothing agent, a muscle relaxant, an antioxidant, a skin conditioner, a skin hydrator, or a skin nourishing agent,
wherein the skincare composition is selected from the group consisting of a body lotion, a face cream, a calming cream, a warming balm, a muscle rub, and combinations thereof.
